(19) United States
(12) Patent Application Publication
(10) Pub. No.: US 2025/0382252 A1
(43) Pub. Date: Dec. 18, 2025

(54) ORGANIC COMPOUNDS

(71) Applicant: Givaudan SA, VERNIER (CH)

(72) Inventors: Lijun Zhou, Shanghai (CN); An Chai, Shanghai (CN); Chun Chen, Shanghai (CN); Changming Ding, Shanghai (CN); Andreas Goeke, Kemptthal (CH); Feng Huang, Shanghai (CN); Marc Liniger, Kemptthal (CH); Zhiyu Yu, Shanghai (CN)

(21) Appl. No.: 18/878,347

(22) PCT Filed: Jun. 28, 2023
(86) PCT No.: PCT/EP2023/067718

§ 371 Date: Dec. 23, 2024

(30) Foreign Application Priority Data
Jun. 29, 2022 (WO) PCT/CN2022/102314

(51) Int. Cl. C07C 49/647; C07C 49/395; C07C 49/597; C11B 9/00

(52) U.S. Cl. CPC C07C 49/647; C07C 49/395; C07C 49/597; C11B 9/003; C07C 2601/08; C07C 2601/10

(57) ABSTRACT

Disclosed are cyclopent-2-enone derivatives possessing woody odor profile with some peppery notes.

### TECHNICAL FIELD

The present invention is concerned with flavor and fragrance ingredients, and to compositions containing them that are useful in flavor and fragrance applications. It furthermore relates to a method of making them. In particular, the present invention is concerned with certain cyclopent-2-enone derivatives of formula (I) possessing powerful woody odor profile with some peppery notes.

### BACKGROUND

In the flavor and fragrance industry, perfumers and flavorists are continually looking for new compounds possessing unique olfactory properties. For example, there is a need for ingredients that are suitable for the flavor and fragrance industry possessing woody odor notes. In particular, there is a need for ingredients possessing woody odor notes with peppery (in particular freshly ground black pepper) aspects.

Shoji Watanabe et al. (Aust. J. Chem., 1982, 35, 1739-41) describes a new convenient preparative method of Jasmone and its related compounds. It is said that the cyclopentenones, which have linear side chains of five or six carbons or with a (Z)-substituted double bond, exhibit a sweet smell like that of jasmone. The only two cyclopentanones compounds disclosed, having a branched side chain, namely 2-(4,4-dimethylpentan-2-yl)-3-methylcyclopent-2-en-1-one and 2-isopropyl-3-methylcyclopent-2-enone, it is mentioned that the later has a fragrance of mint.

GB2100728A relates to the preparation of 3-substituted-2-cyclo-pentenone derivatives, such as 3-(2,6-dimethylheptyl)-2-cyclopentenone, which are suitable as intermediates for the production of compounds possessing insect control properties. As a by-product minor amounts of 3-methyl-2-(6-methylheptan-2-yl)cyclopent-2-en-1-one. However, the patent is silence with regard to any properties of said compound. US2017/0327449 is directed to a Stetter reaction based on the use of bio-sourced reagents and catalysts. Whereas 3-methyl-2-(6-methylhepta-2,5-dien-2-yl)cyclopent-2-en-1-one is mentioned, no further properties are provided.

Surprisingly inventors found a new class of cyclopene-2-enone derivatives of formula (I) as defined herein below possessing the highly sought-after powerful woody odor profile with some peppery notes. When used for the creation of fragrance accords it was surprisingly observed that these compounds possessing sort of transparency although the compounds of formula (I) are very powerful.

### SUMMARY

In accordance with a first aspect of the present invention there is provided the use as fragrance or flavor of a compound of formula (I)

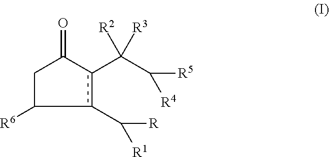

wherein
[symbol] is indicating a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

In accordance with a second aspect of the present invention there is provided a method of improving, enhancing and/or modifying a consumer product base or consumable product base by means of adding thereto an olfactory acceptable amount of a compound of formula (I) or a mixture thereof.

In accordance with a third aspect of the present invention there is provided a composition comprising a compound of formula (I) or a mixture thereof.

In accordance with a fourth aspect of the present invention there is provided a flavour composition comprising a compound of formula (I) or a mixture thereof.

In accordance with a fifth aspect of the present invention there is provided a fragranced article comprising as odorant a compound of formula (I) or a mixture thereof.

In accordance with a sixth aspect of the present invention there is provided a flavoured product comprising as a compound of formula (I) or a mixture thereof.

In accordance with a seventh aspect of the present invention there is provided the use as fragrance a mixture comprising a compound of formula (I) and a compound of formula (II)

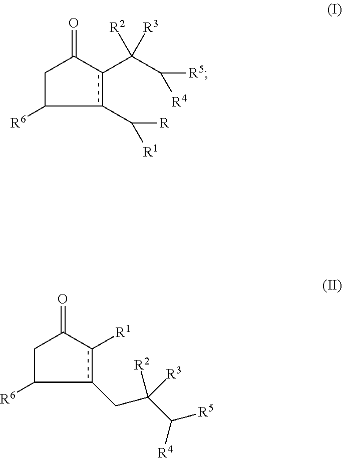

wherein
[symbol] is indicating a carbon-carbon single or double bond,
R is hydrogen,
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

Certain embodiments of any aspect of the present invention may provide one or more of the following advantages:
provision of novel flavour and fragrance compounds with woody odor profile with some peppery notes,
provision of a highly performing compound with low odor threshold.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that cyclopent-2-enone derivatives of formula (I) possessing powerful woody odor profile with some peppery notes.

In a first aspect of the invention, there is provided the use as fragrance or flavour of a compound of formula (I) in the form of any one of its stereoisomers or mixture thereof

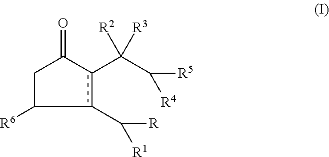

wherein
[symbol] is indicating a carbon-carbon single or double bond
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

In one particular embodiment the compound is a compound of formula (I) wherein R² is methyl and R³ and R⁴ are hydrogen.

In another particular embodiment the compound is a compound of formula (I) wherein R² is methyl and R³ and R⁴ together with the C—C bond represent a double bond.

In another particular embodiment the compound is a compound of formula (I) wherein R² and R³ form together a methylene group and R⁴ is hydrogen.

In another particular embodiment the compound is a compound of formula (I) wherein [symbol] is indicating a carbon-carbon double bond.

In another particular embodiment the compound is a compound of formula (I) wherein R⁵ is selected from C₅ to C₇ linear or branched alkenyl (including C₆ alkenyl) comprising one double bond. For example, R⁵ is selected from the group consisting of pent-2-enyl, 3-methyl (but-2-enyl), 3-methyl (pent-2-enyl), hex-2-enyl, 2-methyl (but-1-enyl), and 2-methyl (but-2-enyl).

In another particular embodiment the compound is a compound of formula (I) wherein R⁵ is selected from C₅ to C₇ linear or branched alkyl (including C₆ alkyl), such as 2-methyl butyl, 3-methyl butyl, pentyl, and hexyl.

In another particular embodiment the compound is a compound of formula (I) wherein R² is methyl, R³ and R⁴ are hydrogen, and R⁵ is selected from C₅ to C₇ linear or branched alkenyl (including C₆ alkenyl) comprising one double bond (e.g. R⁵ is selected from the group consisting of pent-2-enyl, 3-methyl (but-2-enyl), 3-methyl (pent-2-enyl), hex-2-enyl, 2-methyl (but-1-enyl), and 2-methyl (but-2-enyl)).

In another particular embodiment there is provided a compound of formula (I) wherein R² is methyl, R³ and R⁴ are hydrogen, and R⁵ is selected from C₅ to C₆ hydrocarbon residue comprising up to one double bond.

One may cite 3-methyl-2-(nonan-2-yl)cyclopent-2-en-1-one possessing peppery, wood odour profile with some floral, green, metallic, rooty, fenugreek odour characteristics.

As a further specific example one may cite 3-methyl-2-(6-methylhept-5-en-2-yl)cyclopent-2-en-1-one possessing peppery, woody odour profile with some green, floral aspects. It was observed that (R)-3-methyl-2-(6-methylhept-5-en-2-yl)cyclopent-2-en-1-one is about 15 times more powerful than ((S)-3-methyl-2-(6-methylhept-5-en-2-yl)cyclopent-2-en-1-one)-3-methyl-2-(6-methylhept-5-en-2-yl)cyclopent-2-en-1-one. From an olfactory view point the (R) enantiomer was slightly preferred over the(S) enantiomer due to their warm black peppery note, and the patchouli leaves side note.

In another particular embodiment there is provided a mixture comprising a compound of formula (I) wherein R² and R³ form together a methylene group and a compound of formula (I) wherein R³ and R⁴ together with the C—C bond represent a double bond.

Further non-limiting examples are compounds of formula (I) selected from the group consisting of 3-methyl-2-(oct-5-en-2-yl)cyclopent-2-en-1-one; 3-methyl-2-(6-methylocta-2,5-dien-2-yl)cyclopent-2-en-1-one; 3-methyl-2-(6-methylocta-1,5-dien-2-yl)cyclopent-2-en-1-one; 3-methyl-2-(6-methyloct-5-en-2-yl)cyclopent-2-en-1-one; 3-methyl-2-(octa-2,5-dien-2-yl)cyclopent-2-en-1-one; 3-methyl-2-(octa-1,5-dien-2-yl)cyclopent-2-en-1-one; 3-methyl-2-(non-5-en-2-yl)cyclopent-2-en-1-one; 3,4-dimethyl-2-(6-methylhept-5-en-2-yl)cyclopent-2-en-1-one; 3-methyl-2-(5-methylhept-4-en-2-yl)cyclopent-2-en-1-one; 3-methyl-2-(5-methylheptan-2-yl)cyclopent-2-en-1-one; 2-(4,7-dimethyloct-6-en-2-yl)-3-methylcyclopent-2-en-1-one; 2-(6-methylhept-5-en-2-yl)-3-vinylcyclopent-2-en-1-one; 3-ethyl-2-(6-methylhept-5-en-2-yl)cyclopent-2-en-1-one; 3-methyl-2-(6-methylheptan-2-yl)cyclopent-2-en-1-one; 3-ethyl-2-(6-methylheptan-2-yl)cyclopent-2-en-1-one; 3-methyl-2-(octan-2-yl)cyclopent-2-en-1-one; 3-ethyl-2-(oct-5-en-2-yl)cyclopent-2-en-1-one (including (E)-3-ethyl-2-(oct-5-en-2-yl)cyclopent-2-en-1-one); and 2-(oct-5-en-2-yl)-3-vinylcyclopent-2-en-1-one (including (E)-2-(oct-5-en-2-yl)-3-vinylcyclopent-2-en-1-one).

The compounds according to the present invention comprise one or more chiral centres and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methodology known in the art, e.g. preparative HPLC and GC or by stereoselective syntheses.

The compounds of formula (I) may be used alone, as isomeric mixture thereof, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in a mixture with one or more ingredients or excipients conventionally used in conjunction with odorants in fragrance compositions, for example, carrier materials, and other auxiliary agents commonly used in the art.

As used herein, “carrier material” means a material which is practically neutral from an odorant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a fragrance composition. A detailed description of the nature and type of adjuvants commonly used in fragrance compositions containing same cannot be exhaustive, but it has to be mentioned that said ingredients are well known to a person skilled in the art.

As used herein, ‘fragrance composition’ means any composition comprising a compound of formula (I) (which encompasses compounds of formula (Ia)), or a mixture thereof and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following list comprises examples of known odorant molecules, which may be combined with a compound of formula (I), or a mixture thereof:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl) pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione™ (methyl 3-oxo-2-pentylcyclopentaneacetate); 3-(4-isobutyl-2-methylphenyl)propanal; maltol; methyl cedryl ketone; methylionone; verbenone; and/or vanillin;
ether and acetals, e.g. Ambrox® (3a,6,6,9a-tetramethyl-2,4,5,5a,7,8,9,9b-octahydro-1H-benzo [e][1]benzofuran); geranyl methyl ether ((2E)-1-methoxy-3,7-dimethylocta-2,6-diene); and/or Spirambrene® (2′,2′,3,7,7-pentamethylspiro[bicyclo[4.1.0]heptane-2,5′-[1,3]dioxane]);
esters and lactones, e.g. benzyl acetate; cedryl acetate ((1S,6R,8aR)-1,4,4,6-tetramethyloctahydro-1H-5,8a-methanoazulen-6-yl acetate); delta-decalactone (6-pentyltetrahydro-2H-pyran-2-one); Helvetolide® (2-(1-(3,3-dimethylcyclohexyl)ethoxy)-2-methylpropyl propionate); delta-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

Thus there is provided in a further aspect of the invention a fragrance composition comprising a compound of formula (I).

The compounds of formula (I) may be used in a broad range of fragranced articles, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion is typically from 0.00001 to 3 weight per cent of the article. In one embodiment, the compound may be employed in a fabric softener in an amount from 0.0001 to 3 weight per cent (e.g. 0.001 to 1 including 0.5, 0.3, and 0.05 weight %). In another embodiment, the compound may be used in fine perfumery in amounts from 0.01 to 30 weight per cent (e.g. up to about 10 or up to 20 weight per cent), more preferably between 0.01 and 5 weight per cent (e.g. 0.01 to 0.1 weight per cent). However, these values are given only by way of example, since the experienced perfumer may also achieve effects or may create novel accords with lower or higher concentrations.

The compounds of formula (I) may be employed in a consumer product base simply by directly mixing the compound, or a fragrance composition comprising a compound of formula (I), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article, comprising the incorporation a compound of formula (I), or a mixture thereof as a fragrance ingredient, either by directly admixing to the consumer product base or by admixing a fragrance composition comprising a compound of formula (I), or a mixture thereof, which may then be mixed with a consumer product base, using conventional techniques and methods. Through the addition of an olfactory acceptable amount of a compound of formula (I), or a mixture thereof the odor notes of a consumer product base will be improved, enhanced, or modified.

Thus, the invention furthermore provides a method for improving, enhancing or modifying a consumer product base by means of the addition thereto of an olfactorily acceptable amount of a compound of formula (I), or a mixture thereof.

There is provided in a further aspect of the present invention a fragranced article comprising:
a) a compound of formula (I)

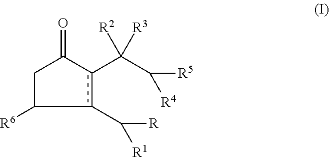

wherein
[symbol] is indicating a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl;
and
b) a consumer product base.

As used herein, ‘consumer product base’ means a composition for use as a consumer product to fulfill specific actions, such as cleaning, softening, and caring or the like. Examples of such products include fine perfumery, e.g. perfume and eau de toilette; fabric care, household products and personal care products such as cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products (includes products that contain preferably volatile and usually pleasant-smelling compounds which advantageously can even in very small amounts mask unpleasant odors). Air fresheners for living areas contain, in particular, natural and synthetic essential oils such as pine needle oils, citrus oil, eucalyptus oil, lavender oil, and the like, in amounts for example of up to 50% by weight. As aerosols they tend to contain smaller amounts of such essential oils, by way of example less than 5% or less than 2% by weight, but additionally include compounds such as acetaldehyde (in particular, <0.5% by weight), isopropyl alcohol (in particular, <5% by weight), mineral oil (in particular, <5% by weight), and propellants.

Cosmetic products include:
(a) cosmetic skincare products, especially bath products, skin washing and cleansing products, skincare products, eye makeup, lip care products, nail care products, intimate care products, foot care products;
(b) cosmetic products with specific effects, especially sunscreens, tanning products, de-pigmenting products, deodorants, antiperspirants, hair removers, and shaving products;
(c) cosmetic dental-care products, especially dental and oral care products, tooth care products, cleaners for dental prostheses, adhesives for dental prostheses; and
(d) cosmetic hair care products, especially hair shampoos, hair care products, hair setting products, hair-shaping products, and hair coloring products.

This list of products is given by way of illustration, and is not to be regarded as being in any way limiting.

In one particular embodiment the consumer product base is selected form fine perfumery, and personal care products, including deodorants, hair care products, soaps, and the like. In one particular embodiment the consumer product base is a hair care product.

In a further particular embodiment the consumer product base is selected from fabric care products, including fabric softener, and home care products, including air fresheners, dish washers and the like.

Whereas certain compounds of formula (I) wherein R⁵ is selected from 3-methyl (butyl) and 3-methyl (but-2-enyl) are described in the literature as intermediates for the preparation of insect growth regulators (Liebigs Ann. Chem. 1986, 509-524), most are novel in their own right.

Thus there is provided in a further aspect of the invention a compound of formula (I)

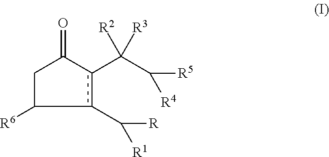

wherein
[symbol] is indicating a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl;
with the proviso that the compound is not a compound of formula (I) wherein R² is methyl, R, R^{1,} R^{3,} R⁴ and R⁶ are hydrogen and R⁵ is selected from 3-methyl (but-2-enyl) and 3-methyl (butyl), and is not 3-methyl-2-(6-methylhepta-2,5-dien-2-yl)cyclopent-2-en-1-one.

The compounds of formula (I) wherein R is hydrogen and R¹ is hydrogen or methyl may, for example, be synthesized by intramolecular aldol condensation of the respective diketone (III) under a base condition (e.g. NaOH) obtaining compounds of formula (I) wherein [symbol] represents a carbon-carbon double bond, optionally followed by hydrogenation to obtain compounds of formula (I) wherein [symbol] represents a carbon-carbon single bond, as depicted below.

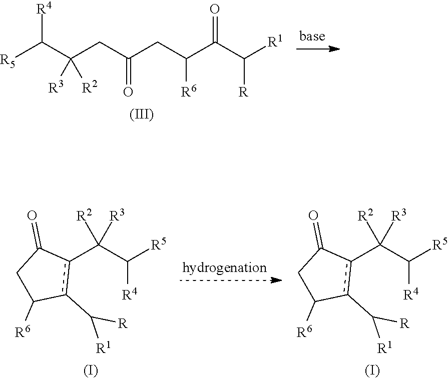

Preparation of the diketones (III) is well known in the art. It may be done, e.g. by 1,4 addition reaction (e.g. Stetter reaction) of the respective aldehyde with the respective α, β-unsaturated ketone in a base condition (e.g. Cs₂CO₃, TEA, sodium L-ascorbate) catalyzed by a thiazolium salt (e.g. thiamine hydrochloride, 3-Ethyl-5-(2-hydroxyethyl)-4-methylthiazolium bromide).

When following this procedure as described above, one may obtain a mixture comprising compounds of formula (I) and compounds of formula (II)

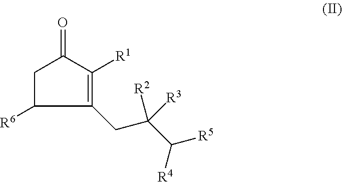

wherein
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

Thus there is provided in a further aspect of the present invention the use as fragrance or flavor of a mixture comprising a compound of formula (I) and a compound of formula (II)

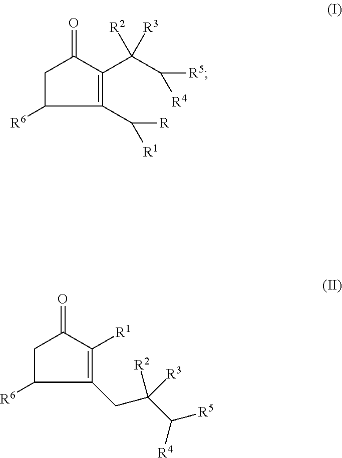

wherein
R is hydrogen,
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

In a further aspect of the present invention there is provide a fragrance composition or a fragranced article comprising a mixture comprising a compound of formula (I) and a compound of formula (II), e.g., in a ratio of 99:1 to about 1:99, such as about 90:10 to about 10:90, which includes a ratio of 80:20 to 20:80, 70:30 to 30:70, 95:5 to 85:15.

Alternatively, the compounds of formula (I) may, for example, be synthesized by in situ addition of 1,3-cyclopentanone with the respective ketone followed by reduction using diethyl 2,6-dimethyl-1,4-dihydropyridine-3,5-dicarboxylate catalyzed by proline. To the thus formed diketone, methanol is added under acidic conditions to form the respective 3-methoxy cyclopentenone derivatives, followed by a Grignard reaction working up under acid conditions.

The invention is now further described with reference to the following non-limiting examples. These examples are for the purpose of illustration only and it is understood that variations and modifications can be made by one skilled in the art.

### Example 1-1: 3-methyl-2-(oct-5-en-2-yl)cyclopent-2-en-1-one

a) A suspension of 3-methylnona-2,6-dienal (11.0 g, 72 mmol) Lindlar Pd (Palladium, 5% on calcium carbonate, lead poisoned, 0.5 g) and potassium carbonate (10.0 g, 72 mmol) in ethyl estate was stirred under H₂ for 28 h. The mixture was filtered through a short silica gel pad, the solvent was removed and the residue oil was purified by column chromatography on silica gel to get 3-methylnon-6-enal (9.0 g, 58 mmol, yield: 81%, E/Z=9:1) as a light yellow oil.
b) A suspension of triethylamine (2.9 g, 29 mmol) and 3-ethyl-5-(2-hydroxyethyl)-4-methylthizolium bromide (2.9 g, 12 mmol) in ethanol (80 mL) was stirred at room temperature for 10 min, and then but-3-en-2-one (8.2 g, 117 mmol) and 3-methylnon-6-enal (9.0 g, 58 mmol) were added. The mixture was refluxed under argon overnight. Water (80 mL) was added, and extracted with MTBE (80 mL*2). The solvent was removed and the residue oil was purified by column chromatography on silica gel to get 7-methyltridec-10-ene-2,5-dione (9.2 g, 41 mmol, yield: 71%, E:Z=9:1) as a light yellow oil.
c) A solution of 7-methyltridec-10-ene-2,5-dione (8.7 g, 38.8 mmol) in ethanol (80 mL) was added an aqueous NaOH solution (0.5 M, 80 mL), the result solution was heated to reflux overnight. MTBE (50 mL*2) was used to extract the solution, the solvent was removed and the residue oil was purified by column chromatography on silica gel to get 3-methyl-2-(oct-5-en-2-yl)cyclopent-2-en-1-one (5.2 g, 25 mmol, yield: 64%, E/Z isomers, E/Z=9:1) as a colorless oil, and 3-(2-methyloct-5-en-1-yl)cyclopent-2-en-1-one (170 mg, 0.8 mmol, yield: 2%, E:Z=6:1) as a colorless oil.
3-methyl-2-(oct-5-en-2-yl)cyclopent-2-en-1-one:
¹H NMR (400 MHz, CDCl₃) δ=5.48-5.20 (m, 2H), 2.74-2.53 (m, 1H), 2.48-2.44 (m, 2H), 2.38-2.25 (m, 2H), 2.04 (s, 3H), 2.01-1.91 (m, 2H), 1.90-1.45 (m, 4H), 1.18-1.09 (m, 3H), 0.94 (t, J=7.5 Hz, 3H) ppm. ¹³C NMR (101 MHz, CDCl_{3,} E isomer) δ=209.5, 169.8, 143.4, 132.1, 129.0, 34.6, 34.0, 31.8, 31.1, 29.8, 25.6, 18.5, 17.5, 13.9 ppm. GC/MS (EI): m/z (%): 206 (11) [M⁺], 191 (5), 177 (3), 150 (18), 124 (100), 109 (22), 67 (21), 55 (19).
Odour description: peppery, woody, floral, fruity, green
3-(2-methyloct-5-en-1-yl)cyclopent-2-en-1-one:
¹H NMR (400 MHz, CDCl_{3,} mixture of isomers) δ=5.94 (s, 1H), 5.54-5.29 (m, 2H), 2.66-1.15 (m, 13H), 1.05-0.86 (m, 6H) ppm. ¹³C NMR (101 MHz, CDCl₃) δ=210.1, 210.1, 182.1, 182.0, 132.4, 132.0, 130.6, 128.6, 128.5, 41.1, 41.0, 36.9, 36.7, 35.3, 31.7, 31.0, 30.9, 29.9, 25.5, 24.5, 20.5, 19.6, 14.3, 13.9 ppm. GC/MS (EI): m/z (%): 206 (3) [M⁺], 191 (5), 177 (3), 163 (4), 149 (14), 123 (100), 110 (61), 96 (35).
Odour description: green, metallic, marine, oyster, oily.

### Example 1-2: 3-methyl-2-(6-methylhept-5-en-2-yl)cyclopent-2-en-1-one

Following the general procedure of Example 1-1, a mixture of 3-methyl-2-(6-methylhept-5-en-2-yl)cyclopent-2-en-1-one and 3-(2,6-dimethylhept-5-en-1-yl)cyclopent-2-en-1-one were obtained from 7,11-dimethyldodec-10-ene-2,5-dione (5.0 g, 22.3 mmol) and sodium hydroxide (0.9 g, 22.3 mmol). 3-Methyl-2-(6-methylhept-5-en-2-yl)cyclopent-2-en-1-one is a colorless liquid (17.4 mmol, 3.6 g, 78% yield) and 3-(2,6-dimethylhept-5-en-1-yl)cyclopent-2-en-1-one is colorless liquid (1.9 mmol, 0.4 g, 9% yield).
3-Methyl-2-(6-methylhept-5-en-2-yl)cyclopent-2-en-1-one:
¹H NMR (400 MHz, CDCl₃) δ=5.15-4.96 (t, J=5.6 Hz, 1H), 2.71-2.52 (m, 1H), 2.50-2.41 (m, 2H), 2.34-2.30 (m, 2H), 2.04 (s, 3H), 1.94-1.69 (m, 3H), 1.66 (s, 3H), 1.58-1.43 (m, 4H), 1.14 (d, J=7.1 Hz, 3H) ppm. ¹³C NMR (101 MHz, CDCl₃) δ=209.5, 169.8, 143.4, 131.3, 124.6, 34.6, 34.2, 31.7, 29.9, 26.6, 25.7, 18.6, 17.6, 17.4 ppm. GC/MS (EI): m/z (%): 206 (22) [M⁺], 191 (3), 150 (15), 137 (21), 124 (100), 109 (23), 67 (22).
Odour description: woody, peppery, green, floral
3-(2,6-dimethylhept-5-en-1-yl)cyclopent-2-en-1-one:
¹H NMR (400 MHz, CDCl₃) δ=5.94 (s, 1H), 5.10-5.06 (t, J=5.1 Hz, 1H), 2.66-1.74 (m, 9H), 1.69 (s, 3H), 1.61 (s, 3H), 1.39-1.20 (m, 2H), 0.92 (d, J=6.6 Hz, 3H) ppm. ¹³C NMR (101 MHz, CDCl₃) δ=210.1, 182.1, 131.7, 130.7, 124.2, 41.1, 36.9, 35.4, 31.7, 31.1, 25.7, 25.4, 19.7, 17.7 ppm. GC/MS (EI): m/z (%): 206 (4) [M⁺], 191 (3), 149 (7), 123 (100), 96 (36).
Odour description: green, citronellic, waxy

### Example 1-3: 3-methyl-2-(6-methylocta-2,5-dien-2-yl)cyclopent-2-en-1-one and 3-methyl-2-(6-methylocta-1,5-dien-2-yl)cyclopent-2-en-1- one

Following the general procedure described in Example 1-1, 7,11-Dimethyltrideca-6,10-diene-2,5-dione was obtained from 3,7-dimethylnona-2,6-dienal (10.0 g, 60.2 mmol) and but-3-en-2-one (4.2 g, 60.2 mmol) as light yellow liquid (13.1 mmol, 3.1 g, 22% yield). A mixture of 3-methyl-2-(6-methylocta-1,5-dien-2-yl)cyclopent-2-en-1-one and 3-methyl-2-(6-methylocta-2,5-dien-2-yl)cyclopent-2-en-1-one were obtained from 7,11-dimethyltrideca-6,10-diene-2,5-dione (3.0 g, 12.7 mmol) and sodium hydroxide (0.5 g, 12.7 mmol). 3-Methyl-2-(6-methylocta-1,5-dien-2-yl)cyclopent-2-en-1-one is colorless liquid (2.4 mmol, 0.5 g, 19% yield, two isomers: 5:7) and 3-methyl-2-(6-methylocta-2,5-dien-2-yl)cyclopent-2-en-1-one is colorless liquid (0.7 g, 3.3 mmol, 26% yield, 4 isomers).
3-methyl-2-(6-methylocta-1,5-dien-2-yl)cyclopent-2-en-1-one:
¹H NMR (400 MHz, CDCl_{3,} mixture of isomers) δ=5.20 (s, 1H), 5.11-4.98 (m, 1H), 4.79 (s, 1H), 2.60-2.29 (m, 6H), 2.08 (s, 3H), 2.05-1.89 (m, 4H), 1.65, 1.55 (s, 3H), 1.00-0.89 (m, 3H) ppm. ¹³C NMR (101 MHz, CDCl_{3,} mixture of isomers) δ=207.9, 207.9, 171.5, 142.2, 142.2, 141.8, 141.7, 137.2, 137.0, 123.5, 122.3, 115.6, 35.5, 35.3, 34.6, 32.2, 31.7, 26.3, 26.0, 24.7, 22.8, 18.0, 18.0, 15.9, 12.7 ppm. GC/MS (EI): m/z (%): 218 (3) [M⁺], 189 (8), 161 (96), 121 (29), 91 (51), 55 (100).
Odour description: woody, peppery, fruity
3-methyl-2-(6-methylocta-2,5-dien-2-yl)cyclopent-2-en-1-one:
¹H NMR (400 MHz, CDCl_{3,} mixture of isomers) δ=5.47-4.81 (m, 2H), 2.83-2.24 (m, 6H), 2.05-1.41 (m, 11H), 0.96-0.73 (m, 3H) ppm. GC/MS (EI): m/z (%): 218 (16) [M⁺], 203 (11), 189 (15), 161 (34), 121 (29), 148 (58), 123 (52), 91 (100).
Odour description: woody, fatty, green, floral, woody.

### Example 1-4: 3-methyl-2-(6-methyloct-5-en-2-yl)cyclopent-2-en-1-one

Following the general procedure described in Example 1-1,
a) 7,11-dimethyltridec-10-ene-2,5-dione (E/Z=4:3) was obtained from 3,7-dimethylnon-6-enal (14.5 g, 86.2 mmol) and but-3-en-2-one (30.2 g, 430.8 mmol) as light yellow liquid (8.1 mmol, 1.9 g, 9% yield, E/Z=4:3).
b) 3-Methyl-2-(6-methyloct-5-en-2-yl)cyclopent-2-en-1-one was obtained from 7,11-dimethyltridec-10-ene-2,5-dione (1.8 g, 7.4 mmol) and sodium hydroxide (0.3 g, 7.4 mmol) as colorless liquid (3.1 mmol, 0.7 g, 42% yield, E/Z=4:3).
¹H NMR (400 MHz, CDCl_{3,} mixture of E/Z isomers) δ=5.08-5.01 (m, 1H), 2.65-2.59 (m, 1H), 2.47-2.45 (m, 2H), 2.33-2.31 (m, 2H), 2.04 (s, 3H), 1.98-1.69 (m, 5H), 1.65, 1.54 (s, 3H), 1.58-1.44 (m, 1H), 1.15-1.13 (m, 3H), 0.98-0.91 (m, 3H) ppm. ¹³C NMR (101 MHz, CDCl_{3,} mixture of E/Z isomers) δ=209.5, 209.5, 169.7, 143.5, 137.1, 137.1, 136.8, 136.8, 124.2, 124.2, 123.0, 123.0, 34.6, 34.6, 34.3, 32.3, 31.7, 29.9, 29.9, 26.4, 26.1, 24.7, 22.9, 18.6, 17.5, 17.4, 15.9, 12.8 ppm. GC/MS (EI): m/z (%): E isomer: 220 (11) [M⁺], 205 (2), 191 (1), 150 (10), 137 (21), 124 (100), 109 (21), 55 (44); Z isomer: 220 (9) [M⁺], 205 (1), 191 (1), 150 (11), 137 (22), 124 (100), 109 (21), 55 (43).
Odour description: woody, peppery, waxy, green

### Example 1-5: 3-methyl-2-(octa-2,5-dien-2-yl)cyclopent-2-en-1-one and 3-methyl-2-(octa-1,5-dien-2-yl)cyclopent-2-en-1-one

Following the general procedure described in Example 1-1,
a) 7-methyltrideca-6,10-diene-2,5-dione was obtained from 3-methylnona-2,6-dienal (15.5 g, 101.8 mmol) and but-3-en-2-one (14.3 g, 203.6 mmol) as light yellow liquid (2.9 g, 13.0 mmol, 13% yield).
b) A mixture of 3-methyl-2-(octa-2,5-dien-2-yl)cyclopent-2-en-1-one and 3-methyl-2-(octa-1,5-dien-2-yl)cyclopent-2-en-1-one was obtained from 7-methyltrideca-6,10-diene-2,5-dione (400 mg, 1.8 mmol) and sodium hydroxide (72 mg, 1.8 mmol). 3-Methyl-2-(octa-2,5-dien-2-yl)cyclopent-2-en-1-one is a colorless liquid (71 mg, 19% yield, mixture of isomers) and 3-methyl-2-(octa-1,5-dien-2-yl)cyclopent-2-en-1-one is a colorless liquid (68 mg, 18% yield, mixture of isomers, E/Z=8:1).
3-Methyl-2-(octa-2,5-dien-2-yl)cyclopent-2-en-1-one (mixture of isomers):
¹H NMR (400 MHz, CDCl_{3,} mixture of isomers) δ 5.64-5.11 (m, 3H), 2.91-2.35 (m, 6H), 2.11-1.92 (m, 5H), 1.85-1.76 (m, 3H), 1.02-0.89 (m, 3H). GC/MS (EI): m/z (%): 204 (31) [M⁺], 189 (20), 175 (35), 161 (35), 147 (55), 135 (55), 119 (55), 105 (70), 91 (100), 77 (70).
Odour description: woody, oily green floral, fruity (peach)
3-Methyl-2-(octa-1,5-dien-2-yl)cyclopent-2-en-1-one (mixture of isomers, E/Z=8:1):
¹H NMR (400 MHz, CDCl_{3,} mixture of E/Z) δ 5.44-5.23 (m, 2H), 5.17-5.08 (m, 1H), 4.81-4.65 (m, 1H), 2.48 (br d, J=4.1 Hz, 2H), 2.42-2.24 (m, 4H), 2.12-1.84 (m, 7H), 0.87 (t, J=7.4 Hz, 3H) ppm. ¹³C NMR (400 MHz, CDCl_{3,} E isomer) δ 208.0, 171.5, 142.3, 141.5, 132.3, 128.3, 115.7, 35.2, 34.6, 31.7, 30.9, 25.5, 18.0, 13.8 ppm. GC/MS (EI): m/z (%): 204 (20) [M⁺], 189 (18), 175 (45), 161 (100), 147 (45), 135 (50), 121 (45), 91 (95), 77 (80).
Odour description: woody, peppery, green floral, fruity

### Example 1-6: 3-methyl-2-(nonan-2-yl)cyclopent-2-en-1-one

a) 3-Methyldeca-2,6-dienal was prepared from 1,1-diethoxy-3-methylbut-2-ene (70.0 g, 389 mmol) and hex-2-en-1-ol (50.7 g, 506 mmol) according to the procedure reported in literature (Helv. Chim. Acta 2017, 100, e1700200) as colorless liquid (33.3 g, 200 mmol, 51% yield).
b) Following the general procedure described in Example 1-1 (step b), 7-methyltetradeca-6,10-diene-2,5-dione was obtained from 3-methyldeca-2,6-dienal (30.0 g, 180 mmol) and but-3-en-2-one (25.3 g, 361 mmol) as light yellow liquid (6.8 g, 29.0 mmol, 16% yield).
c) 7-methyltetradeca-6,10-diene-2,5-dione (4.4 g, 18.6 mmol, 1.0 equiv) and Pd/C (10%, 200 mg) in methanol (50 mL) was hydrogenated for 16h and give 7-methyltetradecane-2,5-dione as light yellow liquid (3.5 g, 14.6 mmol, 78% yield).
d) Following the general procedure described in Example 1-1 (step c), 3-methyl-2-(nonan-2-yl)cyclopent-2-en-1-one was obtained from 7-methyltetradecane-2,5-dione (3.4 g, 14.1 mmol) and sodium hydroxide (0.6 g, 14.1 mmol as colourless liquid (7.3 mmol, 1.6 g, 51% yield).
¹H NMR (400 MHz, CDCl₃) δ=2.67-2.52 (m, 1H), 2.50-2.40 (m, 2H), 2.36-2.25 (m, 2H), 2.05 (s, 3H), 1.69-1.65 (m, 1H), 1.54-1.40 (m, 1H), 1.32-1.10 (m, 14H), 0.87 (t, J=6.9 Hz, 3H) ppm. ¹³C NMR (101 MHz, CDCl₃) δ=209.4, 169.4, 143.7, 34.5, 34.3, 31.9, 31.7, 30.3, 29.6, 29.3, 28.2, 22.6, 18.6, 17.5, 14.1 ppm. GC/MS (EI): m/z (%): 222 (17) [M⁺], 207 (100), 193 (6), 151 (23), 137 (46), 124 (97).
Odour description: peppery, woody, green, metallic, rooty, jasmone, fenugreek.

### Example 1-7: 3-methyl-2-(non-5-en-2-yl)cyclopent-2-en-1-one

Following the general procedure described in Example 1-1,
a) 3-methyldec-6-enal was obtained from 3-methyldeca-2,6-dienal (5.6 g, 33.7 mmol) as colorless liquid (30.0 mmol, 5.0 g, 80% yield, E/Z isomers, E/Z=6:1).
b) 7-methyltetradec-10-ene-2,5-dione was obtained from 3-methyldec-6-enal (4.9 g, 29.1 mmol) and but-3-en-2-one (4.1 g, 58.2 mmol) as light yellow liquid (17.0 mmol, 4.1 g, 59% yield, E/Z isomers, E/Z=3:1).
c) The title compound was obtained from 7-methyltetradec-10-ene-2,5-dione (4.0 g, 16.8 mmol) and sodium hydroxide (0.7 g, 16.8 mmol) as colorless liquid (1.3 g, 6.0 mmol, 36% yield, mixture of E/Z isomers: E/Z=10:1).
¹H NMR (400 MHz, CDCl_{3,} mixture of E/Z isomers) δ=5.47-5.26 (m, 2H), 2.68-2.54 (m, 1H), 2.51-2.43 (m, 2H), 2.35-2.28 (m, 2H), 2.05, 2.04 (s, 3H), 1.98-1.72 (m, 5H), 1.62-1.48 (m, 1H), 1.41-1.27 (m, 2H), 1.17-1.10 (m, 3H), 0.92-0.84 (m, 3H) ppm. ¹³C NMR (101 MHz, CDCl_{3,} mainly Z isomer) δ=209.4, 169.7, 143.4, 130.3, 130.2, 129.8 (Z), 129.7 (Z), 34.7, 34.6, 34.0, 31.7, 31.1, 29.8, 22.7, 18.5, 17.5, 13.6 ppm. GC/MS (EI): m/z (%): 220 (8) [M⁺], 205 (3), 177 (2), 150 (16), 124 (100), 55 (27).
Odour description: woody, green, metallic, rooty, jasmone, fenugreek, peppery, pepperwood

### Example 1-8: 3,4-dimethyl-2-(6-methylhept-5-en-2-yl)cyclopent-2-en-1-one

Following the general procedure described in Example 1-1,
a) 3,7,11-trimethyldodec-10-ene-2,5-dione was obtained from 3,7-dimethyloct-6-enal (10.0 g, 64.8 mmol) and 3-methylbut-3-en-2-one (8.2 g, 97.2 mmol) as light yellow liquid (17.0 mmol, 4.1 g, 27% yield, mixture of isomers: 1:1).
b) 3,4-dimethyl-2-(6-methylhept-5-en-2-yl)cyclopent-2-en-1-one was obtained from 3,7,11-trimethyldodec-10-ene-2,5-dione (4.0 g, 16.8 mmol) and sodium hydroxide (0.7 g, 16.8 mmol) as colorless liquid (10.0 mmol, 2.3 g, 62% yield, mixture of isomers: 1:1).
¹H NMR (400 MHz, CDCl₃ mixture of isomers) δ=5.07 (t, J=9.8 Hz, 1H), 2.74-2.45 (m, 3H), 2.02-1.96 (m, 3H), 1.96-1.69 (m, 4H), 1.68-1.63 (m, 3H), 1.57-1.46 (m, 4H), 1.19-1.10 (m, 6H) ppm. ¹³C NMR (101 MHz, CDCl_{3,} mixture of isomers) δ=208.5, 208.5, 173.8, 173.7, 142.9, 142.8, 131.3, 131.3, 124.6, 124.6, 43.5, 43.4, 37.2, 34.2, 34.2, 30.1, 29.9, 26.6, 26.5, 25.7, 19.0, 17.7, 17.6, 15.0, 14.9 ppm. GC/MS (EI): m/z (%): 220 (46) [M⁺], 205 (2), 177 (5), 164 (8), 151 (20), 138 (82), 123 (100).
Odour description: green, mushroom, milky, woody, cedarwood.

### Example 2-1: 3-methyl-2-(5-methylhept-4-en-2-yl)cyclopent-2-en-1-one

a) A solution of 2-methylfuran (7.4 g, 90.0 mmol) in THF (100 mL) was added butyllithium (75 mmol, 1.6 M in hexane, 50 mL) dropwise at −78° C., and stirred for another hour, and then the reaction solution was warmed up and stirred at r.t. for 4 h. Cool down to −78° C., and 2,5-dimethylhept-4-enal (9.5 g, 67.5 mmol) was added dropwise. The mixture was allowed to warm to rt. in 1 hour after addition and stirred at rt. overnight. The reaction was quenched by diluted NH₄Cl solution (150 mL) at 0° C., and extracted by MTBE (100 mL*2). The combined organic phase was dried over MgSO₄, the solvent was removed by rotary evaporation and the residue oil was purified by column chromatography on silica gel to get 2,5-dimethyl-1-(5-methylfuran-2-yl) hept-4-en-1-ol (10.7 g, 48 mmol, yield: 71%)
b) To a solution of 2,5-dimethyl-1-(5-methylfuran-2-yl) hept-4-en-1-ol (10.5 g, 47.2 mmol) in DCE (200 mL) was added zinc (II) iodide (22.6 g, 70.8 mmol) and sodium cyanoborohydride (22.3 g, 354.2 mmol) at 10° C. The result mixture was stirred at rt. for 1 h. MTBE (200 mL) was used to dilute the reaction mixture, the whole mixture was filtered, and the solvent was removed by rotary evaporation and the residue oil was purified by column chromatography on silica gel to get 2-(2,5-dimethylhept-4-en-1-yl)-5-methylfuran (5.6 g, 27 mmol, yield: 58%, mixture of E/Z isomers, E/Z=7:5) as a colorless liquid.
c) The mixture of 2-(2,5-dimethylhept-4-en-1-yl)-5-methylfuran (5.5 g, 26.7 mmol) and acetic acid (24.0 g, 399.8 mmol) was added 0.2 g con. H₂SO₄. The result solution was heated to reflux for 3 h. Cool down to room temperature and water (50 mL) was added. The result solution was extracted by MTBE (50 mL*2), the combined organic solution was washed with sat. NaHCO₃ solution (30 mL) and brine (20 mL), and dried over MgSO₄. Remove the solvent by rotary evaporation, and the residue oil was purified by column chromatography on silica gel to get 7,10-dimethyldodec-9-ene-2,5-dione and 7,10-dimethyldodec-10-ene-2,5-dione (3.3 g, 14 mmol, 54%) as a brown liquid.
d) A solution of 7,10-dimethyldodec-9-ene-2,5-dione and 7,10-dimethyldodec-10-ene-2,5-dione (2.0 g, 8.9 mmol) and sodium hydroxide (0.4 g, 8.9 mmol) in ethanol (60 mL) was heated to reflux overnight. Cool to room temperature, and water (30 mL) was added, extract by MTBE (60 mL*2), remove the solvent by rotary evaporation and the residue oil was purified by column chromatography on silica gel to get 3-methyl-2-(5-methylhept-4-en-2-yl)cyclopent-2-en-1-one and 3-methyl-2-(5-methylhept-5-en-2-yl)cyclopent-2-en-1-one (1.3 g, 6.1 mmol, yield: 69%) as a colorless oil.
GC/MS (EI): m/z (%): 206 (19) [M⁺], 178 (2), 164 (8), 136 (8), 124 (100), 109 (10), 95 (9), 83 (19), 67 (18), 55 (48) ppm.
Odour description: woody, green, rooty, jasmone, aromatic, coniferous, piney.

### Example 2-2: 3-methyl-2-(5-methylheptan-2-yl)cyclopent-2-en-1-one

a) 7,10-dimethyldodecane-2,5-dione was obtained via hydrogenation of 7,10-dimethyldodec-9-ene-2,5-dione and 7,10-dimethyldodec-10-ene-2,5-dione (1.0 g, 4.4 mmol) catalyzed by Pd/C (10%, 100 mg) in EA (50 mL) as light yellow liquid (0.9 g, 3.9 mmol, 88% yield, mixture of isomers, 1:1).
b) Following the general procedure described in Example 2-1 (step d), 3-methyl-2-(5-methylheptan-2-yl)cyclopent-2-en-1-one was obtained from 7,10-dimethyldodecane-2,5-dione (0.9 g, 4.0 mmol) and sodium hydroxide (0.2 g, 4.0 mmol) as colorless liquid (1.5 mmol, 0.3 g, 39% yield, mixture of isomers: 1:1).
¹H NMR (400 MHz, CDCl_{3,} mixture of two isomers) δ=2.58-2.52 (m, 1H), 2.49-2.45 (m, 2H), 2.36-2.27 (m, 2H), 2.05 (s, 3H), 1.77-1.37 (m, 2H), 1.37-0.80 (m, 14H) ppm. ¹³C NMR (101 MHz, CDCl₃, mixture of two isomers) δ=209.5, 169.5, 169.5, 143.8, 143.7, 35.1, 35.0, 34.5, 34.4, 34.4, 31.8, 31.7, 31.7, 30.6, 30.5, 29.5, 29.3, 19.3, 19.2, 18.7, 18.6, 17.5, 11.4, 11.3 ppm. GC/MS (EI): m/z (%): 208 (5) [M⁺], 193 (19), 179 (5), 151 (42), 125 (100), 109 (31), 79 (29).
Odour description: woody, green, rooty, jasmony, waxy.

### Example 3: 2-(4,7-dimethyloct-6-en-2-yl)-3-methylcyclopent-2-en-1-one

a) To a solution of 2-methylfuran (5.0, 60.9 mmol) in THF (100 mL) was added butyllithium (60.9 mmol, 38 mL, 1.6M in hexane) dropwise over 30 min at −78° C. And after 1 h, the solution was warmed up to rt. and stirred overnight. Then 8-bromo-2,5,7-trimethyloct-2-ene (12.9 g, 55.4 mmol) was added dropwise at 0° C., and then the mixture was stirred at rt. overnight. Water (60 mL) was added to quench the reaction, the mixture was extracted by MTBE (50 mL*2), and the combined organic phase was dried over MgSO₄. The solvent was removed, and the residue was purified by column chromatography on silica gel to get 2-methyl-5-(2,4,7-trimethyloct-6-en-1-yl) furan (6.7 g, 29.0 mmol, yield: 52%) as a light yellow liquid.
b) A solution of 2-methyl-5-(2,4,7-trimethyloct-6-en-1-yl) furan (6.0 g, 25.6 mmol) in 36 mL 75% acetic acid solution (75 g acetic acid and 25 g water and 0.6 g con.H₂SO₄), and then the mixture was heated to reflux for 3 h. After it was cooled to room temperature, water (50 mL) was added, the solution was extracted by MTBE (50 mL*3), and the organic layer was washed with sat. NaHCO₃ solution (20 mL). The solvent was removed by rotary evaporation and the residue oil was purified by column chromatography on silica gel to get 7,9, 12-trimethyltridec-11-ene-2,5-dione (3.5 g, 14.0 mmol, yield: 54%, mixture of two isomers, 1:1) as a light yellow liquid.
c) A solution of 7,9,12-trimethyltridec-11-ene-2,5-dione (3.5 g, 14.0 mmol) and aqueous sodium hydroxide solution (0.6 g, 14.0 mmol) in ethanol (28 mL) was heated to reflux overnight. Water (70 mL) was added to the cooled reaction mixture, MTBE (50 mL*2) was used to extract the solution, the solvent was removed by rotary evaporation and the residue oil was purified by column chromatography on silica gel to get 2-(4,7-dimethyloct-6-en-2-yl)-3-methylcyclopent-2-en-1-one (1.9 g, 8.1 mmol, 58%) as a colorless oil.
¹H NMR (400 MHz, CDCl_{3,} mixture of two isomers) δ=5.19-4.97 (m, 1H), 2.87-2.64 (m, 1H), 2.54-2.38 (m, 2H), 2.36-2.26 (m, 2H), 2.05 (s, 3H), 2.01-1.04 (m, 14H), 0.85-0.80 (m, 3H) ppm. ¹³C NMR (101 MHz, CDCl_{3,} mixture of two isomers) δ=209.5, 209.5, 169.5, 169.2, 144.1, 143.5, 132.0, 132.0, 123.3, 123.1, 41.3, 41.3, 35.8, 35.0, 34.6, 34.5, 32.0, 31.7, 31.7, 31.7, 27.7, 27.7, 25.8, 25.8, 19.7, 19.4, 19.3, 18.5, 17.8, 17.8, 17.5 ppm. GC/MS (EI): m/z (%): 234 (10) [M⁺], 219 (4), 165 (22), 123 (100), 110 (59), 67 (28), 55 (30).
Odour description: woody, green, metallic, fatty, rooty, jasmone, peach, floral, β-ionone.

### Example 4-1: 2-(6-methylhept-5-en-2-yl)-3-vinylcyclopent-2-en-1-one

a) A mixture of cyclopentane-1,3-dione (20.0 g, 204 mmol), L-proline (1.76 g,15 mmol), diethyl 2,6-dimethyl-1,4-dihydropyridine-3,5-dicarboxylate (56.8 g, 224 mmol) and 6-methylhept-5-en-2-one (38.6 g, 306 mmol) in 1,2-dichloroethane (200 mL) was heated to reflux for 48 h. The solvent was removed by rotary evaporation, and the residue was purified by column chromatography on silica gel to get 3-hydroxy-2-(6-methylhept-5-en-2-yl)cyclopent-2-en-1-one (21.0 g, 101 mmol, yield: 49%) as a light yellow solid.
b) A mixture of 2-(6-methylhept-5-en-2-yl)cyclopentane-1,3-dione (8.60 g, 41 mmol), methanol (39.7 g) and 4-methylbenzenesulfonic acid hydrate (0.78 g, 4 mmol) in toluene (150 mL) was refluxed in the presence of a dean-stark to remove water overnight. The mixture was concentrated by rotary evaporation, and the residue was purified by column chromatography on silica gel to get 3-methoxy-2-(6-methylhept-5-en-2-yl)cyclopent-2-en-1-one (7.2 g, 32 mmol, yield: 78%) as a colorless liquid.
c) To a solution of vinylmagnesium bromide (1M in THF, 40 mL) was added 3-methoxy-2-(6-methylhept-5-en-2-yl)cyclopent-2-en-1-one (3.0 g, 13 mmol) in THF (80 mL) dropwise at 0° C. The resulting mixture was stirred at rt. for 3 h. 1.6 M HCl solution (100 mL) was added to quench the reaction at 0° C. The mixture was stirred at rt. for 2 h and extracted with MTBE (50 mL*2). The organic layer was dried over MgSO₄, the solvent was removed and the residue oil was purified by column chromatography on silica gel to get 2-(6-methylhept-5-en-2-yl)-3-vinylcyclopent-2-en-1-one (1.5 g, 6.9 mmol, 53%) as a colorless liquid.
¹H NMR (400 MHz, CDCl₃) δ=6.97 (dd, J=10.8, 17.4 Hz, 1H), 5.72 (dd, J=1.1, 17.3 Hz, 1H), 5.47 (dd, J=1.0, 10.8 Hz, 1H), 5.06-5.02 (m, 1H), 2.80-2.71 (m, 1H), 2.65-2.61 (m, 2H), 2.47-2.31 (m, 2H), 1.90-1.72 (m, 3H), 1.69-1.60 (m, 3H), 1.60-1.45 (m, 4H), 1.18 (d, J=7.0 Hz, 3H) ppm. ¹³C NMR (101 MHz, CDCl₃) δ=209.5, 163.3, 144.5, 131.4, 131.3, 124.4, 120.6, 34.3, 34.1, 29.9, 26.5, 25.7, 24.8, 18.9, 17.6 ppm.
Odour description: peppery, woody, green, metallic, fresh citrus, linalool, bergamot.

### Example 4-2: 3-ethyl-2-(6-methylhept-5-en-2-yl)cyclopent-2-en-1-one

Following the general procedure described in Example 4-1, the title compound was obtained from 3-methoxy-2-(6-methylhept-5-en-2-yl)cyclopent-2-en-1-one (3.0 g, 13.5 mmol) and ethylmagnesium bromide (20 mL, 1.0 M) as colorless liquid (0.5 g, 2.0 mmol, 20% yield).
¹H NMR (400 MHz, CDCl₃) δ=5.04-4.89 (m, 1H), 2.62-2.47 (m, 1H), 2.46-2.29 (m, 4H), 2.29-2.19 (m, 2H), 1.84-1.39 (m, 10H), 1.08-1.04 (m, 6H) ppm. ¹³C NMR (101 MHz, CDCl₃) δ=209.9, 175.0, 142.7, 131.3, 124.6, 34.6, 34.2, 30.0, 28.5, 26.7, 25.7, 24.3, 18.8, 17.6, 12.2 ppm. GC/MS (EI): m/z (%): 220 (25) [M⁺], 205 (2), 191 (12), 163 (4), 138 (100), 123 (22), 109 (29).
Odour description: woody, peppery, green, rooty, stale water, orris.

### Example 4-3: 3-methyl-2-(6-methylheptan-2-yl)cyclopent-2-en-1-one

Following the general procedure described in Example 4-1,
a) 2-(6-methylheptan-2-yl)cyclopentane-1,3-dione was obtained from 2-(6-methylhept-5-en-2-yl)cyclopentane-1,3-dione (10.0 g, 48.0 mmol) as light yellow solid (47.5 mmol, 10.0 g, 99% yield).
b) 3-Methoxy-2-(6-methylheptan-2-yl)cyclopent-2-en-1-one was obtained from 2-(6-methylheptan-2-yl)cyclopentane-1,3-dione (10.0 g, 47.6 mmol) as light yellow liquid (38.4 mmol, 8.6 g, 81% yield).
c) 3-Methyl-2-(6-methylheptan-2-yl)cyclopent-2-en-1-one was obtained from 3-methoxy-2-(6-methylheptan-2-yl)cyclopent-2-en-1-one (1.50 g, 6.7 mmol) and methylmagnesium bromide (4.5 mL, 3.0 M) as colorless liquid (2.0 mmol, 0.42 g, 30% yield).
¹H NMR (400 MHz, CDCl₃) δ=2.68-2.52 (m, 1H), 2.50-2.42 (m, 2H), 2.38-2.27 (m, 2H), 2.05 (s, 3H), 1.68-1.63 (m, 1H), 1.52-1.43 (m, 2H), 1.25-1.04 (m, 7H), 0.83 (q, J=6.6 Hz, 6H) ppm. ¹³C NMR (101 MHz, CDCl₃) δ=209.5, 169.5, 143.7, 38.9, 34.5, 34.5, 31.7, 30.2, 27.8, 25.9, 22.7, 22.5, 18.6, 17.5 ppm. GC/MS (EI): m/z (%): 208 (25) [M⁺], 193 (41), 165 (25), 151 (18), 137 (52), 124 (100), 109 (30).
Odour description: woody, green, jasmone, straw, anisic, metallic, rooty.

### Example 4-4: 3-ethyl-2-(6-methylheptan-2-yl)cyclopent-2-en-1-one

Following the general procedure described in Example 4-1, 3-ethyl-2-(6-methylheptan-2-yl)cyclopent-2-en-1-one was obtained from 3-methoxy-2-(6-methylheptan-2-yl)cyclopent-2-en-1-one (3.0 g, 13.4 mmol) and ethylmagnesium bromide (20 mL, 20.1 mmol, 1.0 M) as colorless liquid (2.5 mmol, 0.56 g, 19% yield).
¹H NMR (400 MHz, CDCl₃) δ=2.59-2.55 (m, 1H), 2.49-2.43 (m, 4H), 2.36-2.27 (m, 2H), 1.72-1.59 (m, 1H), 1.49-1.46 (m, 2H), 1.22-1.04 (m, 10H), 0.83 (dd, J=6.6, 3.0 Hz, 6H) ppm. ¹³C NMR (101 MHz, CDCl₃) δ=209.9, 174.7, 143.0, 38.9, 34.6, 34.5, 30.4, 28.5, 27.9, 26.0, 24.3, 22.7, 22.6, 18.8, 12.2 ppm. GC/MS (EI): m/z (%): 222 (33) [M⁺], 207 (3), 193 (100), 179 (6), 151 (28), 138 (57), 123 (19), 109 (18).
Odour description: woody, green, oily, stale water.

### Example 4-5: 3-methyl-2-(octan-2-yl)cyclopent-2-en-1-one

Following the general procedure described in Example 4-1,
a) 3-hydroxy-2-(octan-2-yl)cyclopent-2-en-1-one was obtained from cyclopentane-1,3-dione (10.0 g, 101.9 mmol) and octan-2-one (39.2 g, 305.8 mmol) as white solid (63.7 mmol, 13.4 g, 63% yield).
b) 3-methoxy-2-(octan-2-yl)cyclopent-2-en-1-one was obtained from 2-(octan-2-yl)cyclopentane-1,3-dione (10.0 g, 47.6 mmol) as light yellow liquid (38.0 mmol, 8.6 g, 81% yield).
c) 3-methyl-2-(octan-2-yl)cyclopent-2-en-1-one was obtained from 3-methoxy-2-(octan-2-yl)cyclopent-2-en-1-one (3.0 g, 13.4 mmol) and methylmagnesium bromide (6.7 mL, 3.0 M) as colorless liquid (2.7 mmol, 0.56 g, 20% yield).
¹H NMR (400 MHz, CDCl₃) δ=2.67-2.51 (m, 1H), 2.51-2.41 (m, 2H), 2.36-2.27 (m, 2H), 2.05 (s, 3H), 1.75-1.58 (m, 1H), 1.56-1.39 (m, 1H), 1.36-1.02 (m, 11H), 0.86 (t, J=6.9 Hz, 3H) ppm. ¹³C NMR (101 MHz, CDCl₃) δ=209.6, 169.6, 143.7, 34.6, 34.3, 31.8, 31.7, 30.3, 29.3, 28.2, 22.7, 18.6, 17.5, 14.1 ppm. GC/MS (EI): m/z (%): 208 (11) [M⁺], 193 (88), 179 (7), 165 (8), 151 (22), 137 (45), 124 (100), 109 (26).
Odour description: green, rooty, fiber, vetiver, woody, spicy, peppery, guayil.

### Example 4-6: (E)-3-ethyl-2-(oct-5-en-2-yl)cyclopent-2-en-1-one

Following the general procedure described in Example 4-1,
a) (E)-3-ethyl-2-(oct-5-en-2-yl)cyclopent-2-en-1-one was obtained from cyclopentane-1,3-dione (27.3 g, 278.1 mmol) and (E)-oct-5-en-2-one (27.0 g, 213.9 mmol) as light yellow solid (10.7 g, 46% yield, purity: 90%).
b) (E)-3-methoxy-2-(oct-5-en-2-yl)cyclopent-2-en-1-one was obtained from (E)-2-(oct-5-en-2-yl)cyclopentane-1,3-dione (10.0 g, 48.0 mmol) as light yellow liquid (29.0 mmol, 6.8 g, 61% yield).
c) (E)-3-ethyl-2-(oct-5-en-2-yl)cyclopent-2-en-1-one was obtained from (E)-3-methoxy-2-(oct-5-en-2-yl)cyclopent-2-en-1-one (6.0 g, 27.0 mmol) and ethylmagnesium bromide (54.0 mL, 1.0 M) as colorless liquid (4.8 mmol, 1.1 g, 18% yield).
¹H NMR (400 MHz, CDCl₃) δ=5.48-5.22 (m, 2H), 2.72-2.54 (m, 1H), 2.46-2.42 (m, 4H), 2.36-2.24 (m, 2H), 2.09-1.91 (m, 2H), 1.91-1.71 (m, 3H), 1.63-1.46 (m, 1H), 1.20-1.07 (m, 6H), 0.94 (t, J=7.5 Hz, 3H) ppm. ¹³C NMR (101 MHz, CDCl₃) δ=209.8, 174.8, 142.7, 132.0, 129.0, 34.6, 34.0, 31.1, 29.9, 28.5, 25.6, 24.6, 18.8, 13.9, 12.2 ppm. GC/MS (EI): m/z (%): 220 (11) [M⁺], 205 (1), 191 (29), 150 (33), 138 (100), 123 (23), 109 (31) ppm.
Odour description: woody, green, violet leaf, undecavertol, soft, transparent, iso e super, sylvamber, suede peppery.

### Example 4-7: (E)-2-(oct-5-en-2-yl)-3-vinylcyclopent-2-en-1-one

Following the general procedure described in Example 4-1, (E)-2-(oct-5-en-2-yl)-3-vinylcyclopent-2-en-1-one was obtained from (E)-3-methoxy-2-(oct-5-en-2-yl)cyclopent-2-en-1-one (2.5 g, 11.2 mmol) and vinylmagnesium bromide (45.0 mL, 1.0 M) as colorless liquid (6.1 mmol, 1.4 g, 54% yield).
¹H NMR (400 MHz, CDCl₃) δ=6.96 (dd, J=10.8, 17.4 Hz, 1H), 5.80-5.22 (m, 4H), 2.83-2.61 (m, 3H), 2.42-2.36 (m, 2H), 2.03-1.52 (m, 6H), 1.18 (d, J=7.1 Hz, 3H), 0.94 (t, J=7.4 Hz, 3H) ppm. ¹³C NMR (101 MHz, CDCl₃) δ=209.5, 163.3, 144.4, 132.3, 131.3, 128.7, 120.6, 34.2, 31.0, 29.8, 25.6, 24.8, 18.8, 13.9 ppm. GC/MS (EI): m/z (%): 218 (13) [M⁺], 203 (20), 189 (26), 149 (82), 136 (91), 121 (75), 91 (100), 77 (71).
Odour description: peppery, woody, green, metallic, rooty, tagetone, jasmine.

### Example 5: 3-methyl-2-(6-methylheptan-2-yl)cyclopentan-1-one

Following the procedure of example 2-2 (step a), 3-methyl-2-(6-methylhept-5-en-2-yl)cyclopent-2-en-1-one (1.5 g, 7.3 mmol) was fully hydrogenated to get 3-methyl-2-(6-methylheptan-2-yl)cyclopentan-1-one (1.2 g, 78% yield) as colorless liquid.
¹³C NMR (101 MHz, CDCl₃, mixture of isomers) δ=221.7, 220.9, 219.9, 219.5, 61.2, 60.8, 59.0, 58.9, 39.3, 39.2, 39.2, 39.0, 35.7, 35.4, 35.4, 35.0, 34.7, 34.4, 33.3, 33.2, 33.0, 32.6, 32.2, 30.2, 30.1, 29.9, 29.8, 28.0, 27.9, 27.9, 25.6, 25.6, 24.7, 24.3, 22.7, 22.7, 22.6, 21.2, 20.5, 17.9, 17.5, 17.0, 16.8, 15.1, 14.9 ppm. GC/MS (EI): m/z (%): 210 (3) [M⁺], 195 (1), 181 (1), 125 (12), 98 (50), 83 (100).
Odour description: peppery, woody, green, fruity.

### Example 6: Fragrance Accord

| Ingredient | parts by weight |
|---|---|
| DECANAL | 0.8 |
| DODECANAL | 0.4 |
| OCTANAL | 0.3 |
| NONANAL | 0.3 |
| ALLYL CAPROATE (2-propenyl hexanoate) | 2 |
| BENZYL ACETATE | 15 |
| BERGAMOT OIL | 120 |
| CEPIONATE (methyl 3-oxo- | 60 |
| 2-pentylcyclopentaneacetate) | |
| CITRONELLOL | 30 |
| CITRONELLYL ACETATE | 10 |
| (3,7-DIMETHYL-6-OCTEN-1-YL ACETATE) | |
| CYCLAL C (2,4-dimethyl-3- | 0.2 |
| cyclohexene-1-carbaldehyde) | |
| A-DAMASCONE(1-(2,6,6-trimethyl- | 2 |
| 2-cyclohexen-1-yl)-2-buten-1-one) | |
| DIHYDRO MYRCENOL | 25 |
| (2,6-dimethyl-7-octen-2-ol) | |
| DIPROPYLENE | 100 |
| GLYCOL (DPG) | |
| HEXENOL-3-CIS | 0.3 |
| ISO E SUPER | 20 |
| ISORALDEINE | 15 |
| LEMON OIL | 200 |
| LINALOOL | 50 |
| LINALYL ACETATE (3,7-dimethyl- | 40 |
| 1,6-octadien-3-yl acetate) | |
| LINDENOL (2-(4-methyl- | 12 |
| 3-cyclohexen-1- | |
| yl)-2-propanol) | |
| LITSEA CUBEBA OIL | 3 |
| ORANGE OIL | 230 |
| PARADISAMIDE (2-ethyl- | 2 |
| n-methyl-n-(3- | |
| methylphenyl)butanamide) | |
| PETALIA (cyclohexylidene- | 3 |
| o-tolyl-acetonitrile) | |
| PHENYL ETHYL | 20 |
| ALCOHOL (2-phenylethanol) | |
| RASPBERRY KETONE | 1 |
| (4-(4-hydroxyphenyl)-2-butanone) | |
| SYLKOLIDE¹ | 15 |
| TRIETHYL CITRATE | 22.68 |
| VANILLIN | 0.02 |
| | 1000 |

1) (E)-2-(3,5-dimethylhex-3-en-2-yloxy)-2-methylpropyl cyclopropanecarboxylate

The fragrance accord above is a fresh citrus cologne accord, e.g., suitable to be applied in alcohol at a dosage of 10 wt % to ethanol (85% vol) which is recognizes as a refreshing citrus cologne.

By replacement of 30 parts DPG with 30 parts of 3-methyl-2-(oct-5-en-2-yl)cyclopent-2-en-1-one results in a accord with more depth and long lastingness. The addition of 3-methyl-2-(oct-5-en-2-yl)cyclopent-2-en-1-one softens the citrus lemon top note and brings a woody ambery cedarwood character with a peppery facet.

1. A method of using a compound of formula (I) or a mixture thereof as a fragrance or flavor 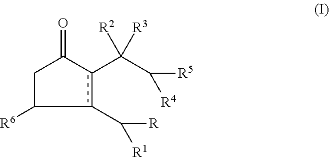
wherein [symbol]
indicates a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl and C₅-C₇ alkenyl, and
R⁶ is selected from hydrogen and methyl.

2. The method according to claim 1 wherein the compound of formula (I) is selected from the group consisting of 3-methyl-2-(oct-5-en-2-yl)cyclopent-2-en-1-one;
3-methyl-2-(6-methylhept-5-en-2-yl)cyclopent-2-en-1-one;
3-methyl-2-(6-methylocta-2,5-dien-2-yl)cyclopent-2-en-1-one;
3-methyl-2-(6-methylocta-1,5-dien-2-yl)cyclopent-2-en-1-one;
3-methyl-2-(6-methyloct-5-en-2-yl)cyclopent-2-en-1-one;
3-methyl-2-(octa-2,5-dien-2-yl)cyclopent-2-en-1-one;
3-methyl-2-(octa-1,5-dien-2-yl)cyclopent-2-en-1-one;
3-methyl-2-(nonan-2-yl)cyclopent-2-en-1-one;
3-methyl-2-(non-5-en-2-yl)cyclopent-2-en-1-one;
3,4-dimethyl-2-(6-methylhept-5-en-2-yl)cyclopent-2-en-1-one;
3-methyl-2-(5-methylhept-4-en-2-yl)cyclopent-2-en-1-one;
3-methyl-2-(5-methylheptan-2-yl)cyclopent-2-en-1-one;
2-(4,7-dimethyloct-6-en-2-yl)-3-methylcyclopent-2-en-1-one;
2-(6-methylhept-5-en-2-yl)-3-vinylcyclopent-2-en-1-one;
3-ethyl-2-(6-methylhept-5-en-2-yl)cyclopent-2-en-1-one;
3-methyl-2-(6-methylheptan-2-yl)cyclopent-2-en-1-one;
3-ethyl-2-(6-methylheptan-2-yl)cyclopent-2-en-1-one;
3-methyl-2-(octan-2-yl)cyclopent-2-en-1-one;
3-ethyl-2-(oct-5-en-2-yl)cyclopent-2-en-1-one;
2-(oct-5-en-2-yl)-3-vinylcyclopent-2-en-1-one and mixtures thereof.

3. The method according to claim 1 wherein the [symbol] indicates a carbon-carbon double bond, and a compound of formula (II) 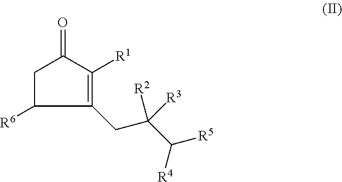
wherein
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl and C₅-C₇ alkenyl, and
R⁶ is selected from hydrogen and methyl.

4. A fragrance composition comprising a compound of formula (I) or a mixture thereof 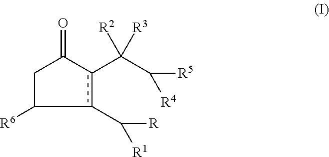
wherein [symbol]
indicates a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl;
and
at least one other odorant.

5. The fragrance composition according to claim 4, wherein the compound is selected from the group consisting of 3-methyl-2-(oct-5-en-2-yl)cyclopent-2-en-1-one; 3-methyl-2-(6-methylocta-2,5-dien-2-yl)cyclopent-2-en-1-one; 3-methyl-2-(6-methylocta-1,5-dien-2-yl)cyclopent-2-en-1-one; 3-methyl-2-(6-methyloct-5-en-2-yl)cyclopent-2-en-1-one; 3-methyl-2-(octa-2,5-dien-2-yl)cyclopent-2-en-1-one; 3-methyl-2-(octa-1,5-dien-2-yl)cyclopent-2-en-1-one; 3-methyl-2-(nonan-2-yl)cyclopent-2-en-1-one; 3-methyl-2-(non-5-en-2-yl)cyclopent-2-en-1-one; 3,4-dimethyl-2-(6-methylhept-5-en-2-yl)cyclopent-2-en-1-one; 3-methyl-2-(5-methylhept-4-en-2-yl)cyclopent-2-en-1-one; 3-methyl-2-(5-methylheptan-2-yl)cyclopent-2-en-1-one; 2-(4,7-dimethyloct-6-en-2-yl)-3-methylcyclopent-2-en-1-one; 2-(6-methylhept-5-en-2-yl)-3-vinylcyclopent-2-en-1-one; 3-ethyl-2-(6-methylhept-5-en-2-yl)cyclopent-2-en-1-one; 3-ethyl-2-(6-methylheptan-2-yl)cyclopent-2-en-1-one; 3-methyl-2-(octan-2-yl)cyclopent-2-en-1-one; 3-ethyl-2-(oct-5-en-2-yl)cyclopent-2-en-1-one; 2-(oct-5-en-2-yl)-3-vinylcyclopent-2-en-1-one and mixtures thereof.

6. A fragranced article comprising
a) as odorant a compound of formula (I) 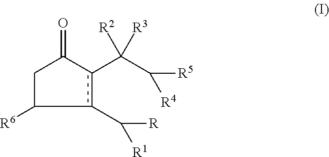
wherein [symbol]
indicates a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl and C₅-C₇ alkenyl, and
R⁶ is selected from hydrogen and methyl;
and
b) a consumer product base.

7. The fragranced article according to claim 6 wherein the odorant compound is a compound of formula (I) where [symbol] indicates a carbon-carbon double bond.

8. The fragranced article according to claim 7 further comprising as odorant a compound of formula (II) 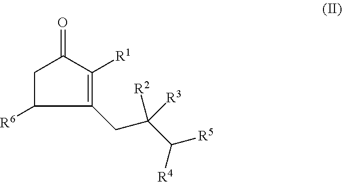
wherein
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl and C₅-C₇ alkenyl, and
R⁶ is selected from hydrogen and methyl.

9. The fragranced article according to claim 6 wherein the consumer product base is selected from fine perfumery, personal care products and fabric care products.

10. A method of improving, enhancing or modifying a consumer product base by means of addition thereto of an olfactory acceptable amount of a compound of formula (I) or a mixture thereof 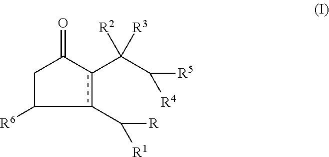
wherein [symbol]
indicates a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from Cs-C₇ alkyl and C₅-C₇ alkenyl, and
R⁶ is selected from hydrogen and methyl.

11. The method according to claim 10 comprising a mixture comprising a compound of formula (I) and a compound of formula (II) 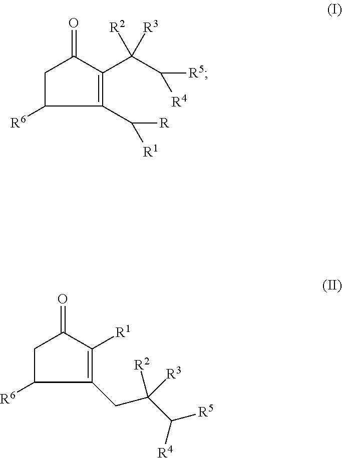
wherein
R is hydrogen,
R¹ is hydrogen or methyl,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl, and C₅-C₇ alkenyl, and
R⁶ is selected from hydrogen and methyl.

12. A compound of formula (I) 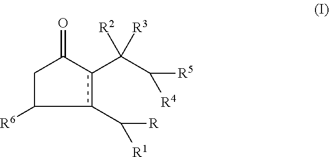
wherein [symbol]
indicates a carbon-carbon single or double bond,
R is hydrogen and R¹ is hydrogen or methyl, or
R and R¹ form together with the C-atom to which they are attached a vinyl group,
R² is selected from methyl and ethyl,
R³ and R⁴ are hydrogen, or
R³ and R⁴ together with the C—C bond represent a double bond, or
R² and R³ form together a methylene group,
R⁵ is selected from C₅-C₇ alkyl and C₅-C₇ alkenyl, and
R⁶ is selected from hydrogen and methyl;
with the proviso that the compound is not a compound of formula (I) wherein R² is methyl, R, R¹, R³, R⁴ and R⁶ are hydrogen and R⁵ is selected from 3-methyl (but-2-enyl) and 3-methyl (butyl), and is not 3-methyl-2-(6-methylhepta-2,5-dien-2-yl)cyclopent-2-en-1-one.

13. The compound according to claim 12 wherein the compound is selected from the group consisting of 3-methyl-2-(oct-5-en-2-yl)cyclopent-2-en-1-one; 3-methyl-2-(6-methylocta-2,5-dien-2-yl)cyclopent-2-en-1-one; 3-methyl-2-(6-methylocta-1,5-dien-2-yl)cyclopent-2-en-1-one; 3-methyl-2-(6-methyloct-5-en-2-yl)cyclopent-2-en-1-one; 3-methyl-2-(octa-2,5-dien-2-yl)cyclopent-2-en-1-one; 3-methyl-2-(octa-1,5-dien-2-yl)cyclopent-2-en-1-one; 3-methyl-2-(nonan-2-yl)cyclopent-2-en-1-one; 3-methyl-2-(non-5-en-2-yl)cyclopent-2-en-1-one; 3,4-dimethyl-2-(6-methylhept-5-en-2-yl)cyclopent-2-en-1-one; 3-methyl-2-(5-methylhept-4-en-2-yl)cyclopent-2-en-1-one; 3-methyl-2-(5-methylheptan-2-yl)cyclopent-2-en-1-one; 2-(4,7-dimethyloct-6-en-2-yl)-3-methylcyclopent-2-en-1-one; 2-(6-methylhept-5-en-2-yl)-3-vinylcyclopent-2-en-1-one; 3-ethyl-2-(6-methylhept-5-en-2-yl)cyclopent-2-en-1-one; 3-ethyl-2-(6-methylheptan-2-yl)cyclopent-2-en-1-one; 3-methyl-2-(octan-2-yl)cyclopent-2-en-1-one; 3-ethyl-2-(oct-5-en-2-yl)cyclopent-2-en-1-one; 2-(oct-5-en-2-yl)-3-vinylcyclopent-2-en-1-one and mixtures thereof.

14. The fragranced article according to claim 8 wherein the consumer product base is selected from fine perfumery, personal care products and fabric care products.
